(19) United States
(12) Patent Application Publication
(10) Pub. No.: US 2007/0276444 A1
(43) Pub. Date: Nov. 29, 2007

(54) Self-powered leadless pacemaker

(76) Inventors: Daniel Gelbart, (US); Samuel Victor Lichtenstein, (US)

Correspondence Address: SEED INTELLECTUAL PROPERTY LAW GROUP PLLC, 701 FIFTH AVE, SUITE 5400, SEATTLE, WA, 98104, US

(21) Appl. No.: 11/439,283

(22) Filed: May 24, 2006

(51) Int. Cl. A61N 1/00

(52) U.S. Cl. 607/6

(57) ABSTRACT

A self-powered pacemaker uses the variations of blood pressure inside the heart or a major artery to create a periodic change in the magnetic flux inside a coil. The pressure variations compress a bellows carrying a magnet moving inside a coil. The inside of the bellows is evacuated to a partial or full vacuum, and a spring restores the bellows to the desired equilibrium point, acting against the blood pressure. The current pulses are stored in a capacitor. Eliminating the battery allows dramatic miniaturization of the pacemaker to the point it can be implanted at the point of desired stimulation via a catheter. The invention includes means of compensating for atmospheric pressure changes.

### BACKGROUND

1. Technical Field

The disclosure relates to self powered medical devices inside the body and in particular to cardiac pacemakers.

2. Description of the Related Art

Cardiac pacemakers are well known, however they have three major shortcomings:
They require major surgery to install and to replace.
They have a limited lifetime because of the battery.
They require running leads from pacemaker to the heart chambers. The leads reduce the reliability of the device and make replacement difficult.

There were many prior attempts to overcome the battery problem by using rechargeable batteries (charged by induction) or electrical energy generated inside the body. To date these attempts were not successful. Rechargeable batteries do not have a longer life than primary batteries at the low power drain of pacemakers (10-50 microwatts), and implanted devices that generate electrical energy were not significantly smaller than the batteries and still required leads. Most reported devices did not generate a sufficient amount of energy. In general, prior attempts to generate electricity from the heart movement or blood pressure can be divided into the following categories:

A. Devices external to the heart, such as US2005/0055061 and UK application GB2350301A.

B. Piezoelectric devices, such as U.S. Pat. No. 4,690,143 and U.S. Pat. No. 4,798,206 and the paper “Self Energized Pacemakers” (Cardiovascular Surgery 1963, supplement to Vol 29, pp 157-160).

C. Inertial devices, such as U.S. Pat. No. 3,554,199; US2004/0073267; U.S. Pat. No. 5,540,729; PCT WO-99/13940; PCT WO-2004/073138; French application 80-06031 (publication number 2 478 996) and JP2000308326A2.

D. Hydraulic devices such as U.S. Pat. No. 3,906,960; U.S. Pat. No. 3,563,245; U.S. Pat. No. RE30366 (re-issue of U.S. Pat. No. 3,835,864); U.S. Pat. No. 3,943,936; U.S. Pat. No. 3,693,625; U.S. Pat. No. 6,827,682 and DE 19535566A1.

The subject matter of the present disclosure belongs to the last group, in which the change in blood pressure is used to generate electricity by moving a magnet relative to a coil. More specifically, the disclosure relates to devices sufficiently small to be implanted at or near the point of desired stimulation, thus avoiding problem associated with leads. Most of the devices in this group (with the exception of U.S. Pat. No. 3,693,625, which relies on tubes and reservoirs located outside the heart) can be potentially located inside the heart and some, such as U.S. Pat. Nos. 3,943,936 and RE30366 even installed by minimally invasive surgery using a catheter percutaneously. However, all patents in this group fail to take into account the very low pressure differentials inside the heart in comparison to atmospheric pressure, thus the energy extracted will be only a small fraction of the estimated power. For example, U.S. Pat. No. RE30366 estimates that the mmHg pressure pulse of the right ventricle will move the transducer 1 mm, generating 130 micro joule of energy (page 8 line 32) while the actual number is only a small fraction of this number. The reason is that any movement of the bellows will increase the air pressure inside the device. In a 1 cm long enclosure, even if the enclosure was completely empty, the movement will only be: 10 mm×20 mmHg/760 mmHg=0.26 mm. When enclosure is filled with the necessary pacemaker electronics, movement is further reduced. In order to achieve high efficiency the transducer has to avoid the increase in internal air (or gas) pressure when its volume is changing. The approaches taught in the present disclosure allow movements of several millimeters from very low pressure changes, with corresponding increases in output power.

A second shortcoming of prior attempts is failing to take into account the effect of high air pressure at high altitudes or inside airplane cabins. The pressure inside an airplane cabin is about 200 mmHg lower than at sea level. This is about 10 times the magnitude of the pressure pulse in the right ventricle. Any device designed to operate on a pressure differential of 20 mmHg and does not take into account an external pressure differential of 200 mmHg is of limited use.

### BRIEF SUMMARY

In one aspect, a self-powered medical device (e.g., pacemaker) is of such small size that it can be implanted at the point of the desired stimulation, thus requiring no leads. The small size also allows percutaneous implantation and replacement, as the device is small enough to fit through the catheters currently used in percutaneous cardiac surgery. If desired, the device can be used with conventional pacing leads. The device can also be used simply as an electrical energy generator inside the body. It can be placed in the heart or in any major artery to supply electricity for devices other than pacemakers, for example de-fibrillators, drug delivery devices, brain stimulators etc. A device having a volume of about a cubic centimeter can supply approximately 30 microwatts continuously. The theoretical possible power output from a one cubic centimeter device placed in the left ventricle of the heart and powered by the blood pressure variation is about 10 mW, thus less than 1% efficiency is required to power a pacemaker. The device may be tolerant to large changes in ambient air pressure without electrical output being affected.

In another aspect, a self-powered medical device uses the variations of blood pressure inside the heart, or a major artery, to create a periodic change in the magnetic flux inside a coil. Typically the pressure variations compress a bellows carrying a magnet moving inside a coil. The inside of the bellows is evacuated to a partial or full vacuum, and a spring restores the bellows to the desired equilibrium point, acting against the blood and atmospheric pressure. The electrical pulses are stored in a capacitor, and used to power the medical device. Since most of the volume of a pacemaker is the battery, eliminating the battery allows dramatic miniaturization of the pacemaker, to the point it can be implanted at the point of desired stimulation. There is no other mechanical coupling to the heart motion except via the changes in blood pressure. This minimizes the interference with the operation of the heart. The compressibility of the device volume with increased pressure is actually an advantage, as it reduces the blood pressure peaks. The device allows for the ambient air pressure to change by allowing the bellows to change length without affecting electrical output.

### BRIEF DESCRIPTION OF THE SEVERAL VIEWS OF THE DRAWINGS

FIG. 1 is an isometric view of the invention according to one illustrated embodiment, drawn with the bellows removed from the base to view the internal parts.

FIG. 2 is a longitudinal cross sectional view of the device of FIG. 1.

FIG. 3 is a graph showing the blood pressure variations required for different implementations.

FIG. 4 is an electrical schematic of the invention according to another illustrated embodiment.

FIG. 5 is a graph of the variations of pressure and voltage in the device.

FIG. 6 is a cross-sectional view of a heart showing possible installation of the device using minimally invasive surgery.

FIG. 7 is a cross-sectional view of the device including a compensation mechanism to compensate for ambient pressure changes.

### DETAILED DESCRIPTION

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Further more, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not interpret the scope or meaning of the embodiments.

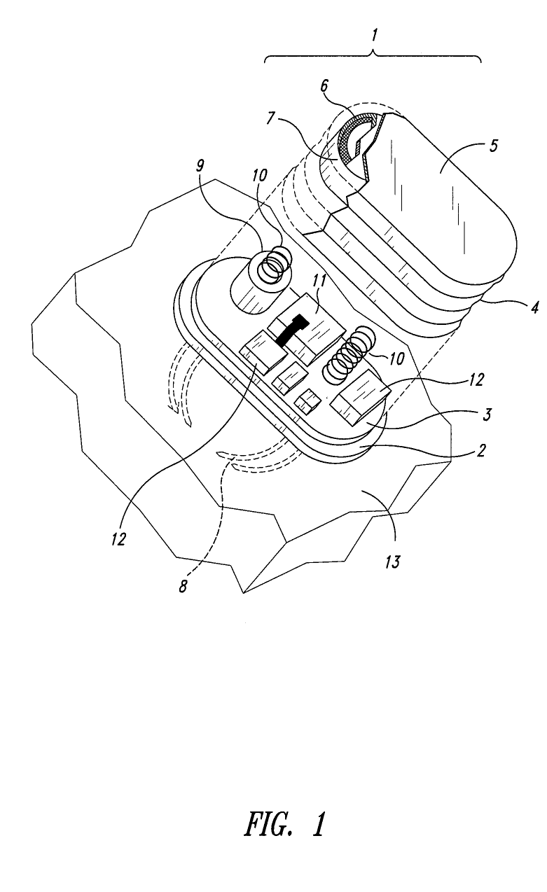

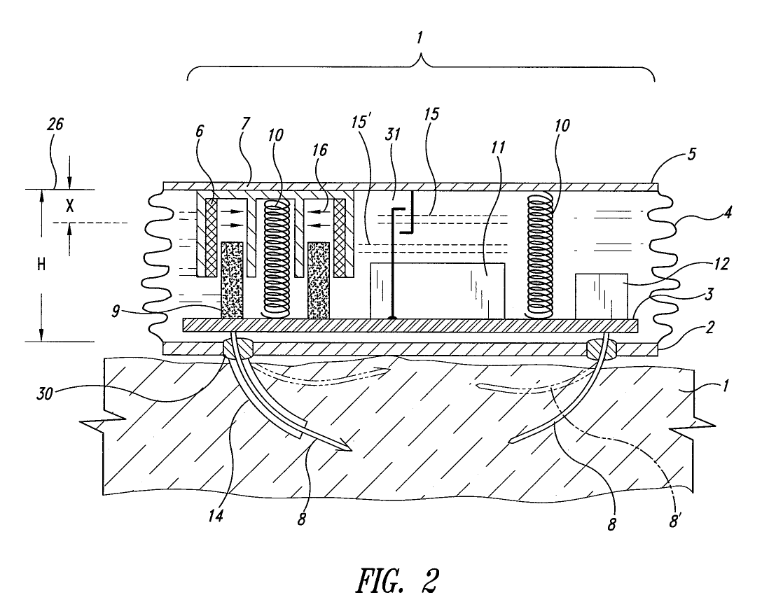

Referring now to FIG. 1, a medical device, for example, a pacemaker 1, comprises of a rigid base 2, an electronics board 3, bellows 4, rigid cover 5, permanent magnet ring 6, ferromagnetic core 7, electrodes 8, coil 9, springs 10, storage capacitor 11 and electronic circuitry 12. Pacemaker 1 is attached to tissue 13 using flexible electrodes 8, which are elastically deformed to pull pacemaker 1 towards tissue 13 and at the same time serve as pacing electrodes. Referring now to FIG. 2, when pacemaker 1 is located inside a heart chamber or major artery, the blood pressure acts on cover 5 and compresses bellows 4. Since blood pressure changes with cardiac movement, cover 5 moves between position shown and position shown by dotted line 15. Total height H, shown by 26, is reduced by amount X. Amount of magnetic field 16 intersecting coil 9 changes with the movement, creating an induced voltage. Electrodes 8 are insulated from base 2 using hermetic seals 30, typically glass-to-metal seals. Bellows 4 is made of metal such as nickel or stainless steel and is welded to base 2, typically by electron-beam welding. The significance of the all metal construction of the enclosure and the hermetic sealing goes beyond the need for reliability. The operation of the device requires that the hermetic seal will be preserved indefinitely, as explained later on. Electronics 12 contains standard pacemaker circuitry and will not be detailed here as it is well known in the art. Electrodes 8 serve both to anchor the pacemaker to the interior of the heart as well as pacing electrodes. It is sometimes desirable to insulate part of the electrode, as shown by 14. Not all electrodes 8 need to be active, some can be used simply for mechanical anchoring and have no electrical function. Some of electrodes 8 can be used as an antenna when pacemaker 1 communicates with external programming devices, or as electrical leads to charge capacitor 11 before installation in body. All standard modes of pace making can be implemented by choosing the number, size and placement of electrodes. In the preferred embodiment the electrodes 8 are made of flexible material such as Nitinol in order to elastically hold pacemaker 1 to the tissue and to be able to flex them when inserted via catheter. The relaxed shape of electrodes 8 is shown by 8′.

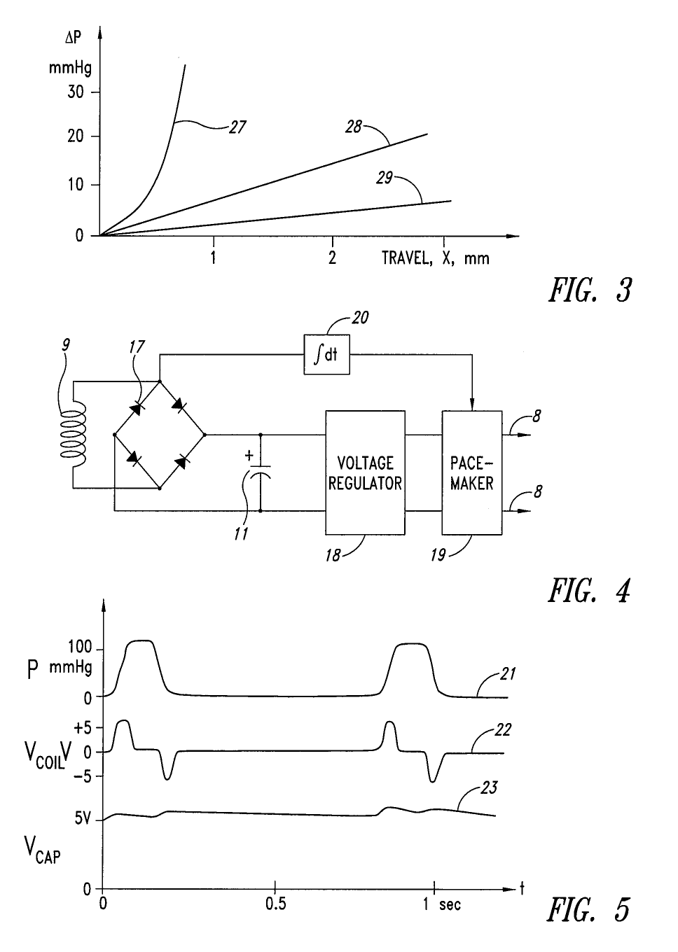

In order to make the size of the device as small as possible the unused internal air space is minimized. This creates a problem, as internal air is compressed when bellows is compressed. The internal air pressure rises as H/(H−x) for an empty case, and much faster if some of the airspace is used. By the way of example, if H in FIG. 2 is 6 mm and half of the internal space is used, leaving an effective H of 3 mm, a blood pressure pulse of 20 mm Hg, as is typical of the right ventricle, will move cover 5 only: x=3 mm×20 mmHg/760 mmHg=0.08 mm. This is insufficient to power a pacemaker. It was found experimentally that a movement of over 1 mm is desired, 3 mm being preferred in a miniature device than can be delivered via a catheter. In the previous example a movement of 1 mm will require a pressure of about 250 mmHg and a movement of 3 mm is not possible, as it will require infinite pressure (since the 3 mm airspace will need to compress to zero volume). This is shown in FIG. 3 graph 27. If the inside of pacemaker 1 is evacuated, the atmospheric pressure plus the blood pressure will always keep the cover pressed down by the maximum amount. The problem is solved by fully or partially evacuating the inside of pacemaker 1 and providing a spring 10 which is always partially compressed. Such a spring restores the position of cover 5 in FIG. 1 to height H, allowing blood pressure to compress it by X. It is desired to choose a spring with a very low spring constant k and a large preload, as seen from the following calculation: Initial length of spring is L, compressed length is H. Force is k(L−H) based on the well known spring formula. The bellows is considered part of the spring constant, or can replace the spring altogether Area of cover 5 is A, and blood pressure changes from a low of P1 to a high of P2 (for example, from 5 to 25 mmHg in the right ventricle).

The values of k and L are derived from the following equations:

A(P1+760 mmHg)=k(L−H)

A(P2+760 mmHg)=k(L−H+X)

By the way of example (substituting 13.6 gm/cm² for every 10 mm Hg):

H=6 mm, x=3 mm, A=2 cm², P1=5 mm Hg (6.8 gm/cm²), P2=25 mmHg (34 gm/cm²)

k(L−6)=2(5+760 mmHg)=2081 gm

k(L−6+3)=2(25+760 mmHg)=2135

Solving for k and L gives k=approx 18 gm/mm and L=approx 122 mm.

Two other forces need to be considered for selecting k:

1. Inertial forces these are small, considering the moving mass is about one gram.

2. Armature reaction force from the interaction of the coil current and the magnetic field.

These are low as well, as the amount of energy extracted per pulse is low.

Since both those forces are proportional to acceleration, it should be verified that they do not slow the rise-time significantly. Since all these forces oppose the blood pressure, the spring constant should be reduced from the calculated value to accommodate these forces. The reason why additional springs are sometimes required is the need to make the wall of the bellows very thin to achieve practically infinite fatigue life. It is important to keep the deformation of the bellows below 20% of its elastic range. Keeping it below 10% is even better. This requires a very thin-walled bellows, which may not have a sufficient k. If some air is left behind inside the device, assuming a partial pressure p, expressed as a fraction of atmospheric pressure (p=1 at 760 mmHg), the equations become:

A(P1+760 mmHg-p760 mmHg)=k(L−H)

A(P2+760 mmHg-p760 mmHg·H/(H−X)=k(L−H+X)

The term (H−X)/H is the increase in p as the volume decreases.

It is clear from the equations that p can only be a very small number before the term pH(H−X) will overpower the effect of the blood pressure, limiting the travel to a very short distance.

Graph 28 and graph 29 in FIG. 2 represent two different values of k. Clearly the spring in graph 29 has a lower k but requires a longer L to achieve a higher initial preload. The limit of how small a pressure difference the device can operate on depends on the desired range of atmospheric pressure changes it will tolerate. Since atmospheric pressure changes can be larger than changes in blood pressure during a cardiac cycle, bellows 9 has to allow movement for atmospheric pressure of about 200 mmHg. For example, the pressure in an airplane cabin can be as low as 560 mmHg. More importantly, the length of coil 9 and magnet 6 had to allow this movement in a manner that some part of the coil is outside the magnetic flux during part of the cardiac cycle, as shown in FIG. 2. At the highest atmospheric pressure cover 5 will level 15′ instead of 15. At the lowest atmospheric pressure the magnetic flux 16 should engage part of coil 9 at the peak of the blood pressure pulse. If the full flux always intersects the coil there will be no induced voltage, as the voltage is only created by the change of flux in the coil. Travel limiter 31 is designed to stop bellows 4 from expanding during transportation, as the device may be subject to atmospheric pressures well below airplane cabin pressure during shipment which could stretch the bellows and damage it. As can be seen, there is a trade-off between the overall height of the device H, the travel X and the atmospheric pressure variations it can operate under. If a small H is desired, a large k spring will have to be used and a small X will result. The power generated is proportional to X. This also means that a device placed in the left atrium or major artery will be more compact than a device placed in the right one, as the blood pressure pulse is 4-5 times larger, allowing k to be larger by the same amount.

In some cases it may be desired to increase the rate of change of the electric flux in order to produce a higher voltage from the coil; for example, when the pacemaker circuitry requires a higher voltage. This can be achieved by adding any one of the known mechanisms to achieve “snap action” to the motion. Typically this is done by using a non-linear spring or by using the inherent non-linearity of magnetic circuits. Placing a small ferromagnetic object on board 3 located near the bottom of the travel will decrease the force towards the end of the travel, since magnet 6 will be attracted downwards. This adds non-linearity to the system and provides a faster rate-of-change of flux. FIG. 4 is an electrical schematic of the pacemaker. The pulses at the output of coil 9 are rectified by rectifier 17, charging capacitor 11 and powering a DC-to-DC converter or simply a voltage regulator 18. In order to use a simple regulator rather than a DC-to-DC converter, coil 9 is wound with very fine magnet wire to generate directly a voltage higher than the voltage required by the pacemaker electronics. The rest of the circuitry is a conventional pacemaker 19, pacing the heart via electrodes 8.

Capacitor 11 can be a tantalum capacitor (to allow reserve power for a few minutes) or a super-capacitor. A super-capacitor will power a pacemaker for many hours without any charging current.

It may be desired to supply the pacemaker electronics 19 with information about blood pressure. Since the voltage in coil 9 is proportional to the derivative of the pressure, is simple to integrate this voltage and re-crate the pressure waveform. This is shown symbolically by integrator 20. The integration can be performed numerically, of course, by the computer controlling the pacemaker functions.

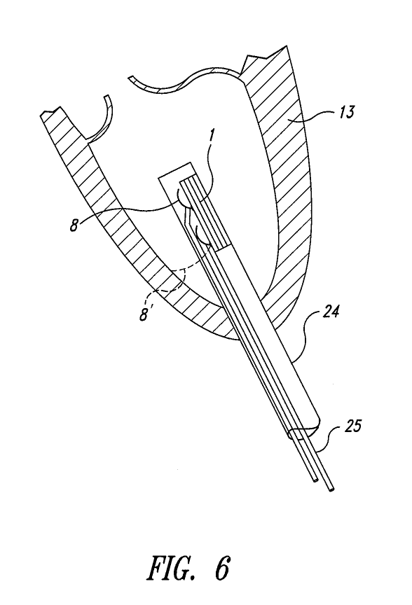

FIG. 5 shows typical waveforms. Graph 21 is the blood pressure in the left ventricle. Graph 21 is the voltage generated across the coil and graph 23 is the voltage across the storage capacitor, with the voltage fluctuations highly exaggerated for clarity. The actual voltage is practically constant, as the capacitor stores the energy of hundreds of pulses. FIG. 6 shows a typical minimally invasive, or percutaneous, deployment of the pacemaker 1 via catheter 24. Tool 25 is used to force the flexible electrodes 8 into position 8′ after pacemaker is pushed out of catheter. At position 8′ the electrodes are pushed into tissue 13 and released. Other catheter based procedures can be used, not requiring piercing a hole in the heart, by entering through the aorta or other major blood vessels. Similar procedures are used to insert the pacemaker 1 into the right ventricle. After a while the pacemaker 1 may become covered with endocardium, which is sufficiently flexible not to interfere with the device operation. If this is not desired, the outside of the pacemaker 1 can be coated with a drug eluting coating or a hydrophobic coating such as thin silicone, or fluorocarbon. While the preferred embodiment is to locate the device inside the arterial blood system, the device can be located outside any major artery, with the cover 5 pressed to the artery wall, and responding to the wall moving with the pressure pulse. Electrodes 8 can be replaced by leads or any other device. All the advantages, such as low operating pressure, are maintained regardless of device being inside or outside the artery wall.

By the way of example, bellows 4 is a 2 cm long×1 cm wide×0.8 cm high custom made bellows made of nickel available from the Servometer Corporation (www.servometer.com). Magnet 6 is a rare-earth ring SmCo magnet with radial magnetization. Core 7 is annealed mild steel. Capacitor 11 is a 680 uF/6.3V surface mount capacitor, 2.8 mm high, from Digikey (www.digikey.com). If a super-capacitor is desired, a 5 mm diameter 0.22 F super-capacitor is available from Cooper Electronic Technology (www.cooperet.com), part number BO510-2R5224. The advantage of a super-capacitor is the ability to deliver a very large amount of power for a short time, as may be needed by some applications. A super-capacitor stores between a 100 to a 1000 fold more energy for the same size as a tantalum capacitor. Base 2 and cover 5 are made of stainless steel, titanium or any other bio-compatible truly hermetic material. A non magnetic material is preferred. Coil 9 is wound with ultra-fine magnet wire such as AWG 56 or 58 available from Wiretron (www.wiretron.com). A prototype device built to these dimensions generated over approximately 30 μW of DC power when operated at a pressure pulse of 100 mmHg, corresponding to being implanted in the left ventricle. Because of the need to maintain a vacuum in the device enclosure for the life of the device, it is important to use construction materials with low outgassing and it is desired to bake the device for a long time and at the maximum temperature allowed before sealing. For example, the device can be baked at 120 deg C. for 100 hours without harming electronic or mechanical components as long as only high temperature polymers are used for internal construction. The exterior, because of the hermetic sealing required, has to be metal with glass-to-metal lead seals. If a polymer exterior is desired (for example, for hydrophobic outside), it should be applied over the metal.

While the description is of a pacemaker, it is obvious the electricity generated can be used for any other purpose in the body and the device can be installed in, or near, any major artery.

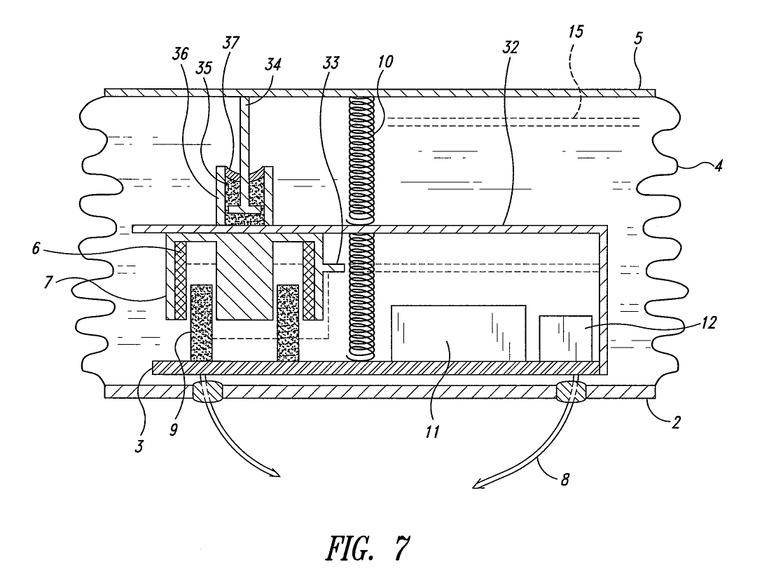

It is possible to add to the device features that compensate for ambient pressure changes in order to keep the coil and magnet to the smallest possible size. This is desired to keep the moving mass (magnet) small and to keep coil inductance minimal. Methods of making devices pressure compensated are well known and they rely on the fact that the ambient pressure changes very slowly (hours) compared to the changes in blood pressure (a fraction of a second). This vast difference in time scale between atmospheric pressure changes and blood pressure changes allows the compensating device to slowly position the coil at the optimal position relative to the moving magnet. An example of a very simple compensating mechanism is shown in FIG. 7. The length of coil 9 and magnet 6 are comparable to the movement caused by blood pressure pulse moving cover 5 to position 15. The slow changes in the position of cover 5 as a function of ambient pressure changes are accommodated by dashpot 36 filled with viscous gel material 35 and having a piston 34 connected to cover 5. Magnet 6 and core 7 are mounted on a leaf spring 32. Slow changes in the position of cover 5 will cause material 35 to flow and accommodate changes. Fast changes, such as blood pressure pulses, will cause spring 32 to follow the motion of cover 5, as material 35 is too viscous to allow fast changes. When cover 5 moves to position 15 at the peak of pressure pulse, spring 32 moves to position 33. All other details are identical to FIG. 2. The viscous material 35 should have non-wetting properties relative to parts 34 and 36, in order to stay contained in dashpot 36, or a seal 37 should be used. If the friction of seal 37 is chosen correctly, no viscous material 35 is required. The advantage of the compensating arrangement of FIG. 7 over the longer coil and magnet of FIG. 2 are less electrical losses in the coil and less moving mass. Clearly in all these examples the positions of the magnet and coil can be reversed; all that matters is the change in magnetic flux through the coil. A third option is to leave both magnet and coil stationary and change the flux by changing the magnetic reluctance of the circuit. Other means of energy generation can be substituted for the magnet and coil, such as piezo-electric generation, which is well detailed in the art of self-powered pacemakers, such as U.S. Pat. No. 4,690,143 and U.S. Pat. No. 4,798,206.

In one aspect, a method for generating electricity from changes in blood pressure, comprises at least partially evacuating a sealed flexible enclosure; subjecting said enclosure to blood pressure changes and creating relative motion between parts of said enclosure; and using said relative motion to create electricity. In another aspect, a method for powering a cardiac pacemaker comprises placing said pacemaker in an at least partially evacuating flexible enclosure; subjecting said enclosure to blood pressure variations for creating relative motion between parts of said enclosure; and using said relative motion to create electricity.

The methods may further include compensating for relative motion not caused by periodic changes in blood pressure. The methods may also include compensating for relative motion caused by atmospheric pressure changes. The methods may also include sensing the blood pressure. The methods may also include the use of a non-linear relationship between blood pressure and said relative motion.

In a further aspect, a cardiac pacemaker deliverable via a catheter, comprises a partially evacuated sealed flexible enclosure that uses flexing of said enclosure for generating electricity. The electricity may be generated by changing the magnetic flux in a coil. Generated electricity may be stored in a capacitor. As noted above, the enclosure may also include a spring, for example a compressed spring. The flexible enclosure may take the form of a metal bellows. In some embodiments, the medical device has outside dimensions of less than 15×15×30 mm.

The pacemakers may have pacing electrodes which may also be used to attach pacemaker to the inside wall of the heart. In some embodiments the pacemaker is placed in the right ventricle of the heart. In some embodiments the pacemaker is placed in the left ventricle of the heart. In some embodiments the enclosure is placed inside the blood circulation system, for example an artery. In still other embodiments the enclosure is placed outside the blood circulation system.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1-20. (canceled)

21. A medical device, comprising:
a flexible enclosure sized to be received in a cardiovascular system of a human, the flexible enclosure forming an inside that is at least a partially evacuated;
a spring biasing the flexible enclosure into an uncompressed configuration; and
a transducer physically coupled to portions of the flexible enclosure to transform relative movement of the portions of the enclosure into electrical power.

22. The medical device of claim 21 wherein the flexible enclosure is a bellows.

23. The medical device of claim 22 wherein the bellows is made of a metal.

24. The medical device of claim 21 wherein the transducer includes a magnet and an electrically conductive coil, the magnet mounted for relative movement with respect to the electrically conductive coil.

25. The medical device of claim 24 wherein the magnet is mounted to transverse longitudinally through at least a portion of the electrically conductive coil.

26. The medical device of claim 21 wherein the spring is positioned in the inside of the flexible enclosure.

27. The medical device of claim 21 wherein the spring is nonlinear.

28. The medical device of claim 21, further comprising:
a circuit board physically coupled to a first end of the flexible enclosure.

29. The medical device of claim 28, further comprising:
a rigid cover physically coupled to seal a second end of the flexible enclosure, opposite the first end of the flexible enclosure.

30. The medical device of claim 21, further comprising:
pacemaker electronics carried by the flexible enclosure and coupled to receive power via the transducer.

31. The medical device of claim 21, further comprising:
a rectifier coupled to the transducer to rectify a current produced by the transducer; and
a voltage regulator coupled to the rectifier to adjust a voltage of the rectified current.

32. The medical device of claim 21, further comprising:
an electrical power storage device electrically coupled to receive power from the transducer.

33. The medical device of claim 32 wherein the electrical power storage device is a super-capacitor.

34. The medical device of claim 21, further comprising:
a travel limiter structure that limits an amount of travel between the portions of the flexible enclosure to compensate for non-periodic changes in ambient pressure.

35. The medical device of claim 21, further comprising:
a computer configured to produce a pulse waveform that is a function of an output of the transducer.

36. A method of making a medical device, the method comprising:
at least partially evacuating an inside of a flexible enclosure that is sized to be delivered via a catheter;
coupling a spring to the flexible enclosure to bias the enclosure into a restored configuration from a compressed configuration;
physically coupling a transducer located in the inside to at least two portions of the flexible enclosure such that the transducer is responsive to relative movement of the flexible enclosure to produce electrical power; and
electrically coupling the transducer to a number of electrodes that extend externally from the flexible enclosure.

37. The method of claim 36, further comprising:
electrically coupling an electrical power storage device to the transducer and the electrodes.

38. The method of claim 36 wherein physically coupling a transducer located in the inside to at least two portions of the flexible enclosure physically coupling a magnet to a first portion of the flexible enclosure and physically coupling an electrically conductive coil to a second portion of the flexible enclosure, the magnet positioned to at least partially extend into the electrically conductive coil.

39. The method of claim 36, further comprising:
physically coupling a circuit board to a first end of the flexible enclosure; and
physically coupling a rigid cover to close a second end of the flexible enclosure, opposite the first end of the flexible enclosure.

40. The medical device of claim 36, further comprising:
electrically coupling a rectifier received in the inside of the flexible enclosure to the transducer to rectify a current produced by the transducer; and
electrically coupling a voltage regulator received in the inside of the flexible enclosure to the rectifier to adjust a voltage of the rectified current.

41. The medical device of claim 36, further comprising:
electrically coupling pacemaker electronics to receive power produced by the transducer.

42. The medical device of claim 36, further comprising:
sealing the at least partially evacuated inside of the flexible enclosure.

43. A method of operating a medical device within at least a portion of a body, the method comprising:
transforming movement of an at least partially evacuated flexible enclosure in response to a blood pressure in the body into an electrical current;
rectifying the electrical current; and
supplying the rectified electrical current to a number of electrodes that extend externally from the flexible enclosure within the portion of the body.

44. The method of claim 43, further comprising:
adjusting a voltage of the rectified electrical current before supplying the rectified electrical current to the electrodes.

45. The method of claim 43, further comprising:
temporarily storing the rectified electrical current before supplying the rectified electrical current to the electrodes.

46. The method of claim 43, further comprising:
compensating for relative motion of the flexible enclosure not caused by changes in blood pressure.

47. A medical device positionable in a body via a catheter, the method comprising:
means for transforming movement of an at least partially evacuated flexible enclosure in response to a blood pressure in the body into an electrical current;
a rectifier electrically coupled to rectify the electrical current; and
a number of electrodes that extend externally from the flexible enclosure within the portion of the body electrically coupled to supply the rectified electrically current to the body.

48. The medical device of claim 47, further comprising:
means for temporarily storing the rectified current electrically coupled o the rectifier.

49. The medical device of claim 47, further comprising:
means for compensating for relative motion of the flexible enclosure not caused by changes in blood pressure.

50. The medical device of claim 47, further comprising:
means for producing a pulse waveform based on a characteristic of the electrical current.
